(19) United States
(12) Patent Application Publication
(10) Pub. No.: US 2011/0189752 A1
(43) Pub. Date: Aug. 4, 2011

(54) COMPLEX

(75) Inventors: Udo Haberl, Baesweiler (DE); Hans -Georg Frank, Kerkrade (NL); Andy Poetgens, Simpelveld (NL); Marco Emgenbroich, Neuss (DE); Andreas Rybka, Gelsenkirchen (DE); Carola Schräder, Neuried (DE); Christoph Kannicht, Berlin (DE)

(73) Assignee: OCTAPHARMA AG

(21) Appl. No.: 12/991,038

(22) PCT Filed: May 6, 2009
(86) PCT No.: PCT/EP09/55503

§ 371 Date: Apr. 26, 2011

(30) Foreign Application Priority Data
May 6, 2008 (EP) 08155718.3

(51) Int. Cl. C12N 9/96; C07K 2/00; C07K 14/755; C07K 14/53; C07K 14/535; C07K 14/54; C07K 14/575; C07K 14/505; C07K 19/00; C08F 8/32; C07K 1/107

(52) U.S. Cl. 435/188; 530/402; 530/383; 530/381; 530/351; 530/399; 530/397; 530/395; 530/409; 530/359; 525/54.1

(57) ABSTRACT

A complex comprising at least one target protein and at least one binding molecule having a binding affinity for said target protein, wherein said molecule having a binding affinity is covalently or non-covalently bound to at least one water-soluble polymer

### FIELD OF THE INVENTION

The present invention relates to complexes of target proteins, binding molecules and polymers. Further disclosed are methods for the preparation of the complexes and use of the complexes.

### BACKGROUND OF THE INVENTION

The use of polypeptides such as proteins for therapeutic applications has expanded in recent years mainly due to advanced knowledge of the molecular biological principles underlying many diseases and the availability of improved recombinant expression and delivery systems for human polypeptides. Polypeptide therapeutics are mainly utilized in diseases where a certain natural polypeptide is defective or missing in the patient, in particular because of inherited gene defects.

For example, hemophilia is a disease caused by deficiency of a certain plasma protein. Hemophiliacs are suffering from hemorrhagic morbidity caused by the disturbed function of protein components of the blood coagulation cascade. Dependent on the affected clotting factor two types of hemophilia can be distinguished. Both have in common the inhibited conversion of soluble fibrinogen to an insoluble fibrin-clot. They are recessive X-chromosomally-linked genetic diseases affecting mainly the male population.

Hemophilia A affects 1-2 individuals per 10.000 males. It is caused by the deficiency or absence of factor VIII, a very large glycoprotein (Mw approximately 330 kDa (Furie B., Furie B. C., Cell (1988) 53, 505-518)), which represents an important element of the blood coagulation cascade. The polypeptide sequence can be subdivided in three regions, an N-terminal region consisting of the so-called A1 and A2-domains, a central B-domain region and a C-terminal region composed of the A3, C1 and C2 domains. In the blood, coagulation factor VIII occurs as an inactive precursor. It is bound tightly and non-covalently to von Willebrand Factor (vWF), which acts as a stabilizing carrier protein. Proteolytic cleavage of factor VIII by thrombin at three specific positions (740, 372, 1689) leads to its dissociation from vWF and releases the procoagulant function within the cascade. In its active form, factor VIII functions as a cofactor for factor IXa, thereby accelerating the proteolytic activation of factor X by several orders of magnitude.

Hemophilia B occurs in about 1 of 25,000 males. It is characterized by the deficiency of the serine protease factor IX (Christmas factor). This 415 amino-acid polypeptide is synthesized in the liver as a 56 kDa glycoprotein. In order to attain its proper function a posttranslational carboxylation step is required which only occurs in the presence of vitamin K.

Treatment of both types of bleeding disorder traditionally involves infusions of human plasma-derived protein concentrates of factor VIII or factor IX. Although this method represents an efficient therapy for hemophiliacs it carries the risk of transmission of various infectious agents, such as viruses causing hepatitis or AIDS, or thromboembolic factors. Alternatively, several recombinant DNA techniques for the production of clotting factors have been described. For this purpose, the corresponding cDNAs of wild type factor VIII and factor IX have been isolated and cloned into suitable expression vectors (EP-A-160457; WO-A-86/01961, U.S. Pat. Nos. 4,770,999, 5,521,070 and 5,521,070).

In the case of factor VIII, recombinant expression of subunits for the production of complexes showing coagulant activity is known in the art (e.g., from EP-A-150735, EP-A-232112, EP-A-0500734, WO-91/07490, WO-95/13300 U.S. Pat. Nos. 5,045,455 and 5,789,203). Moreover, the expression of truncated cDNA-versions partially or entirely lacking the sequence coding for the highly glycosylated B-domain have been described (e.g. in WO-86/06101, WO-87/04187, WO-87/07144, WO-88/00381, WO-94/29471, EP-A-251843, EP-A-253455, EP-A-254076, U.S. Pat. Nos. 4,868,112 and 4,980,456, EP-A-294910, EP-A-265778, EP-A-303540 and WO-91/09122). More recently a variety of selected point mutations have been introduced to inhibit proteolytic inactivation of factor VIII by activated protein C or to reduce the immunogenicity resulting in the formation of inhibitory antibodies by the treated patients (see e.g., U.S. Pat. Nos. 5,859,204, 5,422,260 and 5,451,521, WO-97/49725, WO-99/29848, and M. L. Liu et al., British J. Haematol. 103:1051-1060 (1998)).

However, polypeptide therapeutics such as factor VIII are associated with many drawbacks, including short circulating half-life, immunogenicity and proteolytic degradation. For example, the half-life of the protein factor VIII in the human body is app. 12 hours whereas in severe von Willebrand disease (vWD) patients it is app. 2 hours. Nowadays prophylactic treatment represents the state of the art treatment of hemophilia patients in developed countries. Prophylactic treatment usually results in 2 to 4 infusions per week.

There are a number of further proteins which are used for therapeutic purposes for example erythropoietin, granulocyte-colony stimulating factor (G-CSF), interferons, monoclonal antibodies and the like.

In many cases, it would be helpful to increase the half-life of the therapeutic proteins to increase efficiency or reduce the amount of the therapeutic proteins applied to patient. This would also reduce the costs of the treatment.

In the prior art, the short circulating half-life of polypeptide therapeutics has been addressed by covalent attachment of a polymer to the polypeptide. For example, the attachment of polyethylene glycol (PEG), dextran, or hydroxyethyl starch (HES) has shown some improvement of the half-life of some polypeptides.

However, a number of problems have been observed with the attachment of polymers. For example, the attachment of polymers can lead to decreases in drug activity. Furthermore, certain reagents used for coupling polymers to a protein are insufficiently reactive and therefore require long reaction times during which protein denaturation and/or inactivation can occur. Also, incomplete or non-uniform attachment leads to a mixed population of compounds having differing properties.

One target of the present invention is to overcome drawbacks of prior art, especially to provide an improved method for prolonging the half-life of proteins in the human or animal circulation in order to reduce infusion rates and increase the patient's quality of life.

### SUMMARY OF THE INVENTION

In one embodiment, the present invention is directed to a method for increasing the half-life of a target protein in the circulation of a human or animal.

In one embodiment, the present invention discloses a complex comprising at least one target protein and at least one binding molecule having a binding affinity for said target protein, wherein said molecule having a binding affinity is covalently or non-covalently bound to at least one water-soluble polymer.

Surprisingly, it was found that a non-covalent coupling of a polymer to a target protein via a binding molecule capable of tightly associating to the protein results in a significantly increased half-life revealing a dramatic benefit for the patients.

Due to the association of the binding molecule to a specific binding site of the protein, the polymer coupled to the binding molecule is located in the vicinity of the protein, thus influencing the protein's physiological properties. It is believed that due to the size and physical properties of the polymer, the clearance of the protein from the circulation via the kidneys and/or the degradation of the protein, e.g. via uptake into certain cells, is impaired.

In some cases, the overall size of the formed complex may exceed the maximum size of molecules which can be cleared from the circulation. In particular, the protein bound to the polymer cannot pass the membranes in the kidney and thus, remains in the blood stream and is not excreted via the urine.

The binding molecule and the polymer together form a conjugate and the target protein is non-covalently coupled to said conjugate. Preferably, the binding molecule is coupled covalently to the polymer in said conjugate.

The conjugate may also interfere with the uptake of the protein by cells and/or the recognition and binding of the protein by degrading enzymes. In particular, the conjugate, when bound to the protein, may mask certain protein regions involved in degradation and/or clearance or changing the overall charge of the protein. For example, the conjugate masks binding regions of the protein which are recognized by (i) receptors or factors facilitating the uptake of proteins into cells and/or (ii) proteins involved in the degradation pathways such as proteases, ubiquitin-conjugating enzymes, proteasomes, etc.

Furthermore, the complex and also the methods of the invention provide the advantage that proteins used in therapy do not have to be chemically modified in order to prolong their half-life in the circulation. Rather, the conjugates according to the invention can be incorporated e.g. into the preexisting pharmaceutical composition in order to exert their function. For pharmaceutical applications which rely on clinical approval this is an advantage over conventional coupling strategies, as the therapeutic entity is not altered. The complex according to the present invention is preferably formed ex vivo.

Accordingly, in a first aspect, the present invention provides a complex comprising at least one target protein and at least one binding molecule having a binding affinity for said target protein, wherein said binding molecule having a binding affinity is preferably covalently bound to at least one polymer. The binding molecule and the polymer together form a conjugate which binds to the binding site of the target protein.

In a second aspect, the present invention is directed to the conjugate present in the complex of the first aspect and the use thereof to form a complex.

In a third aspect, the present invention provides a method for preparing a complex according to the first aspect, comprising the step of contacting the target protein with the conjugate.

In a fourth aspect, the present invention provides a method for increasing the half-life of a target protein in the circulation of a human or animal, comprising the step of contacting the target protein with a conjugate comprising a polymer and a binding molecule having a binding capacity to the target protein.

In a fifth aspect, the present invention is directed to the use of the complex according to the first aspect in medicine and for the preparation of a pharmaceutical composition, respectively. Furthermore, this aspect is also directed to a pharmaceutical composition comprising the complex according to the first aspect.

In a sixth aspect, the present invention provides a method for preparing the conjugate according to the third aspect, comprising the step of coupling the polymer to the binding molecule.

Specific embodiments of these aspects of the invention are described hereinafter and in the claims attached hereto.

### BRIEF DESCRIPTION OF THE FIGURES

FIG. 1 shows the three-dimensional structure of blood clotting factor VIII, wherein the interaction sites with low density lipoprotein receptor-related protein (LRP) and heparan sulfate proteoglycan (HSPG) are highlighted.

FIG. 2 shows the structure of the complex of factor VIII with a 1:1 heparinoid-HES conjugate.

FIG. 3 shows a scheme of a target protein with a covalent aptamer-HES conjugate (A=Aptamer).

FIG. 4 shows a scheme for the binding of a posttranslational modified target protein with a mimetic molecule-HES conjugate.

FIG. 5 shows binding studies of HES heparin conjugates using surface plasmon resonance.

FIG. 6 shows the half-life prolongation of factor VIII HES conjugates compared to factor VIII in vitro.

FIG. 7 shows the plasma half-life prolongation of a factor VIII HES conjugate compared to factor VIII in vivo.

FIG. 8 shows surface plasmon resonance based binding studies of human factor VIII and factor IX to fucoidan.

FIG. 9 shows refractive index chromatograms taken from SEC-dRI/UV/MALS measurements of activated HES (C1MaleimideHES—dotted line), thiol modified LMWH (solid line) and a purified LMWH-HES conjugate (dashed line). Column: Superdex200 10/300 GL; mobile phase: 50 mM phosphate buffer pH=6.5 with 150 mM NaCl. (peaks>40 min: salts).

### DETAILED DESCRIPTION OF THE INVENTION

### Definitions

As used herein, the following expressions are generally intended to preferably have the meanings as set forth below, except to the extent that the context in which they are used indicates otherwise.

The expression “comprise”, as used herein, also includes and specifically refers to the expressions “consist essentially of” and “consist of”.

As used herein, the term “complex” particularly means a non-covalent physical association between two or more compounds. The compounds of the complex, here at least the target protein and the conjugate are associated via one or more (non-covalent) intermolecular forces, for example ionic interactions, dipole-dipole interactions, hydrogen bonding, van-der-Waals interactions and/or hydrophobic effects.

The term “conjugate” particularly means two or more compounds which are linked together so that at least some of the properties from each compound are retained in the conjugate. Linking may be achieved by a covalent or non-covalent bond. Preferably, the compounds of the conjugate are linked via a covalent bond. The different compounds of a conjugate may be directly bound to each other via one or more covalent bonds between atoms of the compounds. Alternatively, the compounds may be bound to each other via a linker molecule wherein the linker is covalently attached to atoms of the compounds. If the conjugate is composed of more than two compounds, then these compounds may, for example, be linked in a chain conformation, one compound attached to the next compound, or several compounds each may be attached to one central compound.

As used herein, the term “protein” refers to a molecular chain of amino acids or a complex of more than one amino acid chain. A protein can contain any of the naturally occurring amino acids as well as artificial amino acids and can be of biologic or synthetic origin. A protein may be modified, naturally (post-translational modifications) or synthetically, by e.g. glycosylation, amidation, carboxylation and/or phosphorylation. A protein comprises at least two amino acids, but does not have to be of any specific length; this term does not include any size restrictions. In the present application, the terms “protein”, “polypeptide” and “peptide” are used interchangeably. Preferably, a protein comprises at least 10 amino acids, preferably at least 50 amino acids, at least 100 amino acids and most preferred at least 100 amino acids.

The term “nucleic acid” includes single-stranded and double-stranded nucleic acids and ribonucleic acids as well as deoxyribonucleic acids.

The term “binding site” particularly refers to a region of a protein that, as a result of its shape, hydrophobicity and/or (partial) charge. Favorably, the binding site is naturally capable of associating non-covalently with a target molecule such as e.g. heparin.

Particular examples of binding sites are heparin binding sites capable of binding to heparin and/or closely related compounds such as heparan sulfate, heparinoids and/or derivatives thereof. Another example are metal ion binding sites capable of binding to a metal ion selected from the group consisting of monovalent metal ions, divalent metal ions, trivalent metal ions and tetravalent metal ions. Exemplary metal ions are zinc, copper, cobalt, cadmium and mercury ions. A further example are RGD binding sites capable of binding to RGD peptides, i.e., peptides containing the amino acid sequence arginine (R), glycine (G), aspartate (D), or peptides having a closely related sequence, and/or derivatives thereof. A further example are lipid binding sites capable of binding to lipids including monoglycerides, diglycerides, triglycerides, phospholipids, sphingolipids, saccharolipids, polyketides, sterol lipids, prenol lipids and fatty acids.

As used herein, the term “binding molecule” means a compound capable of non-covalently binding to a binding site of a protein. Preferably, the binding to the binding site is specific, i.e., it is stronger than its interactions with most other molecules present in the natural environment of the protein, e.g. the human or animal circulation. In preferred embodiments, the binding moiety binds to the binding site with an affinity having a dissociation constant under physiologic conditions of 1 mM or less, more preferably 300 μM or less, 100 μM or less, 30 μM or less, 10 μM or less, 3 μM or less, 1 μM or less, 300 nM or less, 100 nM or less, 30 nM or less and most preferably 10 nM or 1 nM or less. The structure and composition of the binding moiety is not restricted in any way as long as it is able to bind to the binding site. Preferably the binding moiety is non-toxic and physiological acceptable. For example, the binding moiety may be a peptide moiety, saccharide moietiy, nucleic acid moiety, lipid moiety or a mimetic compound thereof, or a combination thereof. Particular examples of binding molecule are heparin, heparan sulfate, heparin-like molecules such as in particular heparin-mimetic peptides, RGD peptides, metal ion mimetics, and lipids, derivatives thereof and mimetics thereof.

The term “polymer” particularly refers to a compound that is composed of two or more molecular species (“monomers”) forming a covalently bonded larger molecule. Polymers may be natural or synthetic and linear, branched or dendritic. The polymer may be further derivatized with suitable substituents such as reactive groups.

The term “water-soluble polymer” refers to a polymer which is soluble in water. In contrast to solid polymers, which are used to produce for example micro titer plates, tubes and the like. At a concentration of 1 mg/ml the water-soluble polymer forms a solution with water without phase separation.

The term “hydroxyalkyl starch” or “HAS” refers to a starch derivative which is substituted by at least one hydroxyalkyl group. The starch preferably is naturally occurring starch like potato starch or maize starch. The hydroxyalkyl groups preferably are bound to the oxygen at the C2, C3 and/or C6 carbon atom of the glucose units and mainly occur at C2 and C6. The amount of introduced hydroxyalkyl groups may be expressed as the molar degree of substitution MS, which is defined as the average number of hydroxyalkyl groups per glucose molecule (maximal MS=3.0; usually between 0.4 and 0.7). The attached hydroxyalkyl groups may have any suitable chemical structure but preferably are straight chain or branched lower alkyl groups having, for example, 1 to 10 carbon atoms. Preferably, the hydroxyalkyl group has 2 to 8, and more preferably 2 to 4 carbon atoms. For example, the hydroxyalkyl group may be a hydroxyethyl, hydroxypropyl or hydroxybutyl group, with hydroxyethyl, in particular 2-hydroxyethyl being most preferred. Hydroxyalkyl starch wherein the hydroxyalkyl group is hydroxyethyl, preferably 2-hydroxyethyl, is termed “hydroxyethyl starch” or “HES”.

A “derivative” of a compound particularly refers to said compound being substituted by one or more chemical moieties and/or wherein one or more chemical moieties have been deleted. Derivatives include those that are obtained by naturally occurring processes (e.g., phosphorylation of proteins, methylation of nucleic acids, etc.) as well as those obtained by chemical synthesis. Exemplary substituents of a derivative are functional groups such as hydroxy, carboxy, keto, aldehyde, amino, sulfite, sulfate and phosphate groups; or other groups such as alkyl, aryl, alkoxy, etc.

A “mimetic” of a target compound, as used herein, particularly means a compound that has one or more properties which are similar to the properties of the target compound. In particular, if the target compound is capable of binding to a specific binding site of a protein, the mimetic of the target compound, e.g. heparin, is also able to bind to said binding site, preferably with a comparable affinity. More preferable, the dissociation constant of the mimetic, defining the affinity to the binding site, is at most 1000 times the dissociation constant of the target compound, more preferably at most 500 times, at most 100 times, at most 50 times, at most 20 times, at most 10 times, at most 5 times, at most 2 times, at most 1.5 times, at most 1.2 times the dissociation constant of the target compound, and most preferably it is equal to or lower than the dissociation constant of the target compound.

In a preferred embodiment, the three-dimensional structure and/or the spatial distribution of charge of a mimetic resembles that of the target compound—at least in the portion, or a part thereof, relevant for binding to the binding site.

A “linker” is a chemical moiety linking two or more target compounds to each other. To this end, the linker molecule, before it is coupled to the target compounds, exhibits functional groups capable of forming covalent bonds with the target compounds. The link between two target compounds may be formed using one linker molecule, or a sequence of linker molecules may be attached to each other before the second (or both) target molecules are coupled.

The term “circulation”, as used herein, particularly refers to the cardiovascular system and/or the lymphatic system of a human or animal, preferably the cardiovascular system, including the heart, blood and the blood vessels.

The “half-life” of a compound particularly refers to the time required for the amount of the compound to decay to half of its initial value. For example, the half-life of a compound in circulation is the time required for the concentration of the compound in circulation to decrease to half of the initial concentration, e.g. the concentration at the time the compound has been added to the circulation. The half-life of a compound in circulation is influenced by a variety of factors such as the renal clearing rate of the compound, the rate of (enzymatic) degradation of the compound, the rate of uptake of the compound into cells, etc.

The term “pharmaceutical composition” particularly refers to a composition suitable for administering to a human or animal, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

The term “molecular weight” of a compound particularly refers to the weight of one mol of said compound. If the compound is polydisperse, the molecular weight refers to the weight-average molecular weight of the polydisperse mixture which, thus, may include single compounds having a higher or lower molecular weight than the indicated weight-average molecular weight.

All patents, patent applications, scientific articles and other documents mentioned herein are incorporated herein by reference.

The present invention is based on the finding that the half-life of a target protein in the human or animal circulation can be increased by non-covalently binding a polymer to the target protein using a binding molecule that is coupled to the polymer.

In this respect, the invention is directed to a complex comprising a target protein and a conjugate comprising a polymeric moiety and a binding moiety having a binding capacity to the target protein, wherein the target protein is non-covalently coupled to the conjugate.

The target protein whose circulating half-life is to be increased may be any protein as long as it exhibits a binding site capable of associating with the binding moiety. Preferably, the protein is soluble in water. In certain embodiments, the protein has a pharmacological activity in human and/or animals and preferably is useful in the treatment of a disease, for example a disease as discussed below. Preferably, the target protein is directly administered to the circulation in the treatment, e.g. by intravenal or intraarterial injection.

In preferred embodiments, the target protein has therapeutic activity. Suitable target proteins comprise binding sites selected from the group consisting of a heparin binding site, an RGD peptide binding site, an RGD motif, a metal ion binding site, a lipid binding site and a nucleic acid binding site.

Suitable target proteins are selected from blood clotting proteins such as factor IX, factor VIII (wild-type and B-domain deleted), Factor VII/VIIa, thrombin, antithrombin, tissue plasminogen activator and von Willebrand factor (vWF), growth factors such as erythropoietin, colony-stimulating factors (CSFs) such as granulocyte stimulating factor (G-CSF), macrophage CSF (M-CSF) and granulocyte-macrophage CSF (GM-CSF), cytokines such as interleukins, protease inhibitors such as alpha-1-antitrypsin (A1AT), integrins, disintegrins, extracellular matrix proteins such as fibronectin and vitroncectin, metalloproteases such as matrix metalloproteases and ADAM/ADAMTS proteins, metalloproteases, apolipoproteins, transport proteins, hormones, inhibitory or regulatory acting proteins, and derivatives and mutants thereof.

In some embodiments, the target protein has a molecular weight of 10 kDa or more, preferably 30 kDa or more, and most preferably 50 kDa or more. Furthermore, the protein may have a molecular weight of 1000 kDa or less, preferably 500 kDa or less, most preferably 200 kDa or less.

Suitable target proteins comprising a heparin binding site are, for example, antithrombin, thrombin, von Willebrand factor, tissue plasminogen activator, factor VIII, factor IX, vitronectin, protein C inhibitor, tissue factor pathway inhibitor, platelet factor 4, histidine-rich glycoprotein, thrombospondin, urokinase, fibronectin, fibroblast growth factors, hepatocyte growth factor, lipases, apolipoprotein B, apolipoprotein E.

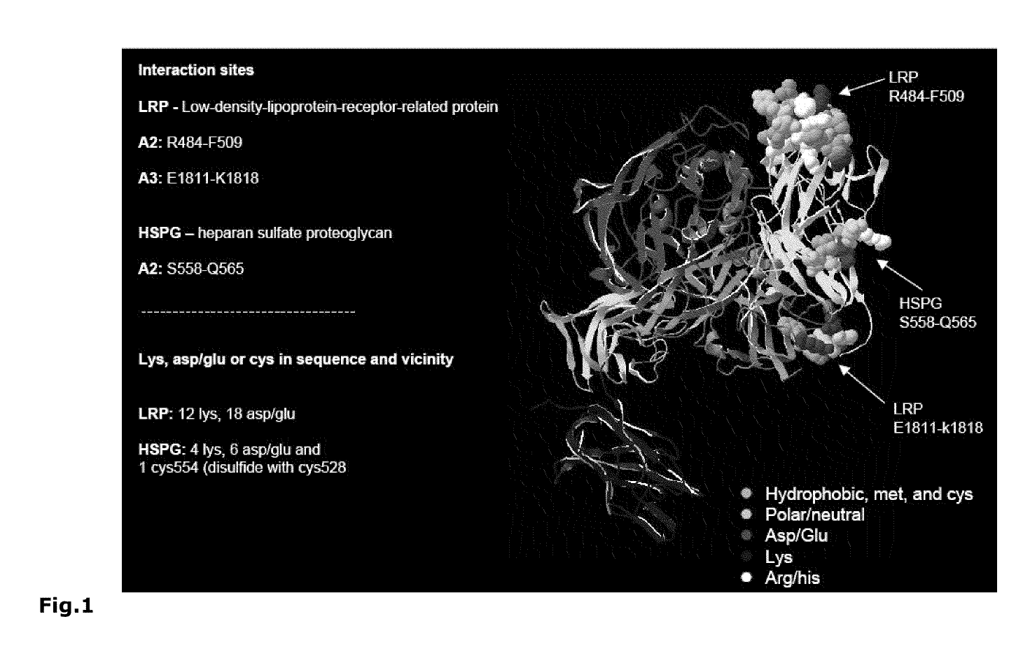

Examples of binding sites suitable for the invention are heparin binding sites, RGD peptide binding sites, RGD motifs, Src homology 2 (SH2) domains, Src homology 3 (SH3) domains, metal ion binding sites, lipid binding sites nucleic acid binding sites. In a preferred embodiment, the protein comprises a heparin binding site such as that of blood clotting factor VIII shown in FIG. 1.

Target proteins comprising an RGD binding site include, for example, integrins. Examples of RGD motif containing target proteins are disintegrins and extracellular matrix proteins such as fibronectin and vitronectin.

Target proteins comprising a metal ion binding site include, for example, metalloproteases such as matrix metalloproteases and ADAM/ADAMTS proteins; and metallotheoneins.

Target proteins comprising a lipid binding site include, for example, apolipoproteins.

In preferred embodiments, the target protein comprises a binding site for the binding moiety that at least partially participates in the degradation and/or clearance of the target protein.

Depending on the target protein whose half-life in circulation is to be increased, the binding moiety of the conjugate has to be selected. Preferably, the binding moiety binds selectively to the target protein. Normally, the complex of the target protein and the conjugate is formed in vitro in the absence of other proteins capable of binding to the binding moiety.

A strong affinity and specificity of the binding moiety to the target protein is preferred in order to assure that the complex remains for a certain time period intact, i.e. associated upon administration. This is important in order to achieve the prolongation of the half-life. The binding strength of the interaction of two compounds usually is expressed by its dissociation constant Kd. The dissociation constant Kd is an equilibrium constant, which describes the ratio of unbound compounds to bound compounds, e.g. the concentrations of free target protein and free conjugate to the protein-conjugate complex. The typical range in biological systems is between micro- and picomolar. For example, under physiological salt concentrations, the Kd value of heparin-binding to proteins (i.e. heparin-binding proteins) is in the low to high nanomolar range. For example Olson et al., 1981 J. Biol. Chem., 256, 11073-11079 determined a dissociation equilibrium constant of 7.2+/−1.9×10⁻⁸ M for heparin binding to antithrombin III, which demonstrates the high affinity of protein-heparinoid interactions.

Thus, the dissociation constant Kd of the target protein-binding moiety interaction and likewise, of the target protein-conjugate interaction, preferably is in the micromolar range or below. Preferably, said dissociation constant is 100 μM or lower, more preferably 100 nM or lower.

The binding molecule may be of any chemical nature including peptide moieties, saccharide moieties, nucleic acid moieties, lipid moieties and other organic compounds.

In one embodiment, the binding molecule comprises the natural ligand of the target protein, or a part of the ligand capable of binding to the target protein. However, the binding molecule does not have to comprise the natural ligand of the target protein. In one embodiment, the binding molecule comprises a mimetic of the natural ligand of the target protein. Such mimetics may be of any chemical nature, and preferably are peptide mimetics comprising natural and/or artificial amino acids; saccharide mimetics comprising natural and/or artificial monosaccharides; nucleic acid mimetics comprising natural and/or artificial nucleotides or peptide nucleic acids coupled via normal phosphodiester linkages or artificial linkages; or organic compound mimetics.

Thus, in case the target protein comprises a heparin binding site, the binding molecule may be a heparin moiety, a heparan sulfate moiety or a heparin-like moiety such as fucoidans, sulphated fucans or heparinoids, i.e. highly acidic, e.g. highly sulphated polysaccharides, or a mimetic thereof. The heparin can be a low molecular weight heparin (LMWH) having a weight-average molecular weight of less than about 8000 Da, such as enoxaparin (Clexan; Mw=4.2 kDa) or the fully synthetic pentasaccharide Fondaparinux (Arixtra; 1726.77 g/mol). Preferably, the heparin, heparan sulfate or heparinoid is in average composed of from 3 to 20 monosaccharide units, more preferably from 5 to 15 monosaccharide units, such as pentasaccharides.

Preferably, the heparin-like molecule is a peptide binding to the heparin binding site of the target protein such as a heparin mimetic peptide. Thus, the mimetic of heparin or heparan sulfate may be a peptide preferably comprising negatively charged amino acids like Glu or Asp, amino acids modified with a negatively charged group such as sulfated amino acids, in particular sulfated tyrosine, and phosphorylated amino acids, in particular phosphorylated tyrosine, serine or threonine, and/or non-natural amino acids with acid moieties in the side chain. Preferably, the heparin mimetic peptide comprises one or more sulfated amino acids such as sulfated tyrosine. In one embodiment, the mimetic peptide comprises the amino acid motif X¹—Y(SO₃)—X²—Y(SO₃), wherein Y(SO₃) is a sulfated tyrosine, X¹ is a negatively charged amino acid, serine, alanine or glycine and X² is an aspartate, alanine or is absent. Exemplary mimetic peptides comprise the amino acid motif:

SY(SO₃)DY(SO₃),

SY(SO₃)DY(SO₃)SY(SO₃)DY(SO₃) or

Y(SO₃)Y(SO₃)GGY(SO₃)DY(SO₃).

Furthermore, the mimetic of heparin or heparan sulfate may be a chemical compound such as suramin or derivatives thereof; organic polymers such as sulfonated polymers, polymers with amino acid side chains and polymers with mono- or disaccharide side chains; or sulfated saccharides, in particular sulfated pentasaccharides.

Suitable mimetics of heparin are described, for example, in H. H. A. M. Hassan, Mini-Reviews in Medicinal Chemistry 7:1206-1235 (2007), S. H. Kim and K. L. Kiick, Peptides 28:2125-2136 (2007), and H. D. Maynard and J. A. Hubbell, Acta Biomateriala 1:451-459 (2005), herein incorporated by reference.

If the target protein comprises an RGD binding site, the binding moiety may be a natural or synthetic peptide comprising an RGD motif. Suitable sequences can be found in integrin binding proteins such as disintegrins and extracellular matrix proteins, e.g. fibronectin and vitronectin.

If the target protein comprises an RGD motif, the binding moiety may be an RGD binding peptide such as an RGD binding protein domain as found, for example, in several integrins, or a synthetic compound specifically binding to the RGD motif, for example a cyclic peptide as described in WO 90/03983 or WO 97/08203.

In case the target protein comprises an SH2 domain, the binding moiety may be an SH2 binding peptide which preferably comprises a tyrosine residue, more preferably a phosphorylated tyrosine residue, or a mimetic of an SH2 binding peptide.

In case the target protein comprises an SH3 domain, the binding moiety may be an SH3 binding peptide which preferably comprises a proline residue, or a mimetic of an SH3 binding peptide. Preferably, the SH3 binding peptide comprises the amino acid motif P-X-X-P wherein P is proline and X is any amino acid, preferably an aliphatic amino acid; or the amino acid motif R-X-X-K with R being arginine, K being lysine and X being any amino acid.

In case the target protein comprises a metal ion binding site the binding molecule may be a ligand of a metal complex such as an amine, a carboxylate or a thiol.

In case the target protein comprises a lipid binding site, the binding moiety may be a lipid such as a phospholipid or a fatty acid.

If the target protein comprises a nucleic acid binding site, the binding moiety may be a naturally occurring or synthetic nucleic acid, such as a single-stranded or double-stranded ribonucleic acid or deoxyribonucleic acid, or a mimetic thereof, such as a peptide nucleic acid. If the nucleic acid binding site is sequence specific, the binding moiety preferably is a nucleic acid having the required sequence or a closely related sequence, such as a sequence having about 80%, 85%, 90%, 95% or 98% sequence identity with the entire sequence of the binding motif.

According to one embodiment, the binding molecule is not an antibody or a part of an antibody comprising a variable region or a binding part thereof. According to a further embodiment, the target protein is not an antibody or a part of an antibody comprising a variable region or a binding part thereof. Furthermore, according to one embodiment neither the binding molecule nor the target protein is an antibody or a part of an antibody comprising a variable region or a binding part thereof.

In a preferred embodiment, the target protein is a heparin binding site-comprising protein such as factor VIII or factor 1× and the binding moiety is heparin, heparan sulfate, a heparinoid, or a mimetic thereof.

To minimize the influence of the binding molecule on the function of the target protein, the binding moiety preferably should be as small as possible. Thus, the binding moiety preferably has a molecular weight of 50 kDa or less, more preferably 10 kDa or less, even more preferably 5 kDa or less. Furthermore, the binding molecule may have a molecular weight of 100 Da or more, for example 500 Da or more.

The increase in half-life of the target protein is mainly attributed to the attachment of the conjugate. Thus, the conjugate, when bound to the target protein, preferably increases the half-life of the target protein in the circulation of a human or animal body. For example, the conjugate, when bound to the target protein, may be capable of interfering with the clearance of the target protein from the circulation or with the degradation of the target protein and/or masks one or more regions of the target protein involved in the uptake of the target protein by cells and/or the recognition and binding of the target protein by degrading enzymes.

The polymer comprises at least one natural or synthetic linear, branched or dendritic polymer and preferably is soluble in water and body fluids and more preferably is hydrophilic. In preferred embodiments, the polymeric moiety is biologically inert and/or pharmaceutically acceptable.

The polymer may be of any size suitable for the desired purpose. In view of this, the polymer preferably has a molecular weight of 5 kDa or more, more preferably 10 kDa or more. As upper limit, the polymer preferably has a molecular weight of 1000 kDa or less, more preferably 300 kDa or less, and most preferably 200 kDa or less.

In certain embodiments, the polymer comprises a polymer selected from the group consisting of polysaccharides, polypeptides, nucleic acids, polyethers, polyesters and polyolefines. Preferably, the polymeric moiety comprises a polymer selected from the group consisting of polyalkylene glycol and derivatives thereof, including polyethylene glycol (PEG), PEG homopolymers, mPEG, polypropyleneglycol homopolymers, copolymers of ethylene glycol with propylene glycol, wherein said homopolymers and copolymers are unsubstituted or substituted at one end e.g. with an acylgroup; polyglycerines or polysialic acid; hydroxypropyl methacrylate (HPMA) and its copolymers; polyglutamate, carbohydrates, cellulose and cellulose derivatives, including methylcellulose and carboxymethylcellulose; starches such as hydroxyalkyl starch (HAS), especially hydroxyethyl starch (HES) and dextrines, and derivatives thereof; dextran and dextran derivatives, including dextransulfat, carboxymethyl dextran, crosslinked dextrin, and carboxymethyl dextrin; chitosan, polyvinyl alcohol and polyvinyl ethyl ethers; polyvinylpyrrollidon; alpha,beta-poly[(2-hydroxyethyl)-DL-aspartamide; and polyoxyethylated polyols.

In a certain embodiment, the polymer comprises hydroxyalkyl starch, preferably hydroxyethyl starch, having an average molecular weight (Mw) of at least 5 kDa and preferably, maximal 1000 kDa, more preferably between 8 and 500 kDa and even more preferably between 10 and 300 ka and 100 to 300 kDa. It may have a molar degree of hydroxyalkyl (hydroxyethyl) substitutions per glucose unit of from about 0.4 to about 1.3, preferably 0.4 to 0.9 or 0.4 to 0.7.

The polymer may be directly coupled to the binding moiety. In case the polymeric moiety and/or the binding moiety do not possess an appropriate coupling group, one or more linkers can be used to appropriately modify the polymer and/or binding molecule in order that they can react with at least one reactive group on the other molecule to form the conjugate. To this end, any suitable coupling strategy known in the art can be used. Suitable linkers, polymeric moeities and conjugation methods are also described in detail in WO 2007/101698 and Orlando, Michele, Dissertation, Modification of proteins and low molecular weight substances with hydroxyethyl starch (HES), Justus-Liebig-Universitat Giessen, Germany, 2003, herein incorporated by reference. Preferably, the coupling is specific such that only one or a defined number of reactive groups of the binding molecule and/or the polymer is used for the coupling. For the coupling of the polymer, the binding molecule and optionally one or more linkers to each other, any suitable reactive groups can be used.

Thus, if the binding molecule or polymer comprises a peptide, the coupling reaction may specifically be directed to the alpha-chain amino or carboxy group or to the side chain reactive group of an amino acid which preferably occurs only once in the peptide. Examples of suitable side chain reactive groups are the cysteine thiol group, the lysine, arginine or histidine amino group, the aspartate or glutamate carboxy group, the asparagine or glutamine amido group, and the serine, threonine or thyrosine hydroxy group. However, also reactive groups introduced by artificial amino acids may be used for coupling.

In case the binding molecule or the polymer comprises a peptide, the following reactive groups present on or introduced to the other moiety may be used for coupling:
acylating groups which react with amino groups of a peptide, for example acid anhydride groups, N-acylimidazole groups, azide groups, N-carboxy anhydride groups, diketene groups, dialkyl pyrocarbonate groups, imidoester groups, and carbodiimide-activated carboxyl-groups. All of the above groups are known to react with amino groups on e.g. proteins to form covalent bonds, involving acyl or similar linkages;
alkylating groups which react with sulfhydryl (mercapto), thiomethyl, imidazo or amino groups of a peptide, such as halo-carboxyl groups, maleimide groups, activated vinyl groups, ethylenimine groups, aryl halide groups, 2-hydroxy-5-nitro-benzyl bromide groups; and aliphatic aldehyde and ketone groups together with reducing agents, reacting with amino groups of a peptide;
ester and amide forming groups which react with carboxyl groups of a peptide, such as diazocarboxylate groups, and carbodiimide and amine groups together;
disulfide forming groups which react with the sulfhydryl groups of a peptide, such as 5,5′-dithiobis (2-nitrobenzoate) groups, ortho-pyridyl disulfides and alkylmercaptan groups which react with sulfhydryl groups in the presence of oxidizing agents such as iodine;
dicarbonyl groups, such as cyclohexandione groups, and other 1,2-diketone groups which react with guanidine moieties of a peptide;
diazo groups, which react with phenolic groups of a peptide;
reactive groups from reaction of cyanogen bromide with a polysaccharide, which react with amino groups of a peptide.

If a suitable amino acid for performing the coupling reaction is missing in a binding/polymeric moiety comprising a peptide, the respective amino acid may be introduced into the peptide, preferably at the amino- or carboxy-terminal end thereof.

If the binding moiety and/or the polymeric moiety comprise a saccharide, the coupling reaction may specifically be directed to the aldehyde/hemiacetal group or the keto/hemiketal group, respectively, on the reducing end of the saccharide. Suitable coupling strategies are described, for example, in WO 2004/024776 A1.

For example, the aldehyde/hemiacetal group at the reducing end may be subjected to a reductive amination with a primary amino group present on or introduced to the other moiety. The reaction is preferably carried out in the presence of a reducing agent such as boron hydrides (e.g. sodium cyanoborohydride, sodium triacetoxyborohydride, sodium borohydride) or organic boron complexes. Alternatively, the aldehyde/hemiacetal group may be subjected to a selective oxidation using an oxidizing agent such as iodine, bromine or a suitable metal ion, or electrochemical oxidation, resulting in a carboxy group or activated carboxy group such as an ester, lactone or amide. The (activated) carboxy group may then be reacted with an amino group, e.g. in the presence of an activating agent such as N-hydroxysuccinimide, N-hydroxyphthalimide, thiophenol, p-nitrophenol, o,p-dinitrophenol, trichlorophenol, trifluorophenol, pentachlorophenol, pentafluorophenol, 1-hydroxy-1H-benzotriazole (HOBt), 3-hydroxy-1,2,3-benzotriazine-4(3H)-one (HOOBt), 4-hydroxy-3-nitrobenzene sulfonic acid (HNSA), 2-hydroxypyridine, 3-hydroxypyridine, 3,4-dihydro-4-oxobenzotriazine-3-ol, 4-hydroxy-2,5-diphenyl-3(2H)-thiophenone-1,1-dioxide, 3-phenyl-1-(p-nitrophenyl)-2-pyrazoline-5-on), [1-benzotriazolyl-N-oxy-tris(dimethylamino)phosphonium hexafluorophosphate] (BOP), [1-benzotriazolyloxytris(pyrrolidino)phosphonium hexafluorophosphate (PyBOP), [O-(benzotriazol-1-yl)-N,N,N′,N′-tetramethyluronium hexa-fluorophosphate (HBTU), 2-(1H-7-azabenzotriazol-1-yl)-N,N,N′,N′-tetramethyluronium hexafluorophosphate (HATU), [O-(benzotriazol-1-yl)-N,N,N′,N′-tetramethyluronium tetrafluoroborate (TBTU), [O-(benzotriazol-1-yl)-N,N,N′,N′-bis(pentamethylene)uronium hexafluorophosphate, [O-(benzotriazol-1-yl)-N,N,N′,N′-bis(tetramethylene)uronium hexafluorophosphate, carbonyldiimidazole (CDI), or carbodiimides, e.g. 1-(3-dimethylaminopropyl)-3-ethylcarbodiimide (EDC), dicyclohexylcarbodiimide (DCC), or diisopropylcarbodiimide (DIPC).

If the binding moiety and/or the polymeric moiety comprise a nucleic acid, the coupling may be made via a 2′-hydroxy group, a 3′-hydroxy group, or a 5′-hydroxy group. If the binding moiety comprises a fatty acid, the carboxy group thereof may be used. If the polymeric moiety comprises an organic polymer such as a polyester, polyether or polyolefin, the reactive group at the end of the polymer—if present—, a reactive group of a monomeric unit of the polymer or an introduced reactive group may be used for coupling.

Thus, the conjugate according to the invention may be made by—optionally—first modifying the polymeric moiety and/or the binding moiety chemically to produce a polymeric moiety and/or binding moiety having at least one chemical group thereon which is capable of reacting with an available or introduced chemical group on the other moiety, and then reacting together the polymeric moiety and the binding moiety to form a covalently bonded conjugate thereof. Alternatively, the optionally modified polymeric moiety or the optionally modified binding moiety may first be coupled to a (further) linker and then the linker may be coupled to the optionally modified other moiety.

In a specific embodiment of the coupling reaction, a first linker is coupled to the polymeric moiety, wherein said first linker bears a reactive group which does not react with the polymeric moiety and which is not present in the polymeric moiety. A second linker is coupled to the binding moiety, wherein said second linker bears a reactive group which does not react with the binding moiety and which is not present in the binding moiety. Then a third linker is coupled either successively or simultaneously to the first and to the second linker. The reactive groups used for coupling the third linker to the first and second linker may be any reactive groups suitable for this purpose. Furthermore, during the coupling of the first and second linker to the polymeric moiety and binding moiety, respectively, the reactive groups of the linker may be protected by a protecting group.

Exemplary reactive groups, which may be used for coupling the polymeric moiety, the binding moiety and optionally one or more linkers to each other, are C—C-double bonds or C—C-triple bonds or aromatic C—C-bonds, acyl halide, halo, hydroxy, aldehyde, amido, amino, aminooxy, hydroxyamino, carbonyl, carboxy, activated ester, cyano, thiocyano, imino, nitro, nitroso, nitrile, peroxy, phospho, sulfonyl, sulfinyl, thiol, hydrazine, and hydrazide groups. The pairs of reactive groups useful for coupling the linkers to each other are, for example, an amino group and a succinimide ester group, a thiol group and a maleimide group.

Examples of protecting groups which may be used in the coupling reactions are:
for the protection of alcohols: acetyl (Ac), β-methoxyethoxymethyl ether (MEM), methoxymethyl ether (MOM), p-methoxybenzyl ether (PMB), methylthiomethyl ether, pivaloyl (Piv), tetrahydropyran (THP), silyl ether such as trimethylsilyl (TMS), tert-butyldimethylsilyl (TBDMS), and triisopropylsilyl (TIPS) ethers, methyl ethers, and ethoxyethyl ethers (EE);
for the protection of amines: carbobenzyloxy (Cbz), p-methoxybenzyl carbonyl (Moz or MeOZ), tert-butyloxycarbonyl (BOC), 9-fluorenylmethyloxycarbonyl (FMOC), benzyl (Bn), p-methoxybenzyl (PMB), 3,4-dimethoxybenzyl (DMPM), p-methoxyphenyl (PMP), tosyl (Ts), and sulfonamides (Nosyl & Nps);
for the protection of carbonyl groups: acetals, ketals, acylals and dithianes; and
for the protection of carboxylic acids: methyl esters, benzyl esters, tert-butyl esters and silyl esters.

The number of binding molecules and the number of polymers in the conjugate may be freely chosen as fits. Thus, the conjugate may comprise one or more binding molecule such as only one, two or more, three or more and four or more. Likewise, the conjugate may comprise one or more polymers such as only one, two or more, three or more and four or more. In one embodiment, the conjugate comprises one binding molecule and one polymer.

If linkers are used for coupling the binding molecules to the polymer, one linker may couple two molecules to each other, e.g. one binding moiety to one polymeric moiety, or one linker may couple more than two molecule to each other, e.g. one binding moiety to two or more polymers, two or more binding molecules to one polymer, or two or more binding molecules to two or more polymers.

The linker may have any suitable length, but preferably is as short as possible. However, the linker preferably is long enough so that the polymeric moiety does not interfere with the interaction of the binding moiety and the target protein. Suitable linkers for coupling molecules are well-known in the prior art and are also commercially available.

A suitable molecule for forming the linker in the conjugate comprises two or more functional groups (as described above) for coupling the linker to the binding molecule, the polymer and/or another linker molecule. These functional groups are preferably connected via a pharmaceutically acceptable bridging group such as those derived from aliphatic or aromatic carbohydrates. The linker may be homo- or hetero bifunctional.

The entire conjugate may have any size suitable for the desired application. However, the conjugate preferably has a molecular weight of 5 to 1000 kDa, preferably 20 to 300 kDa.

The complex of the invention may be formed of one or more, e.g. one, two, three, four or more, target proteins and one or more, e.g. one, two, three, four or more, conjugates. In one embodiment, the complex comprises only one target protein and only one conjugate.

The half-life of the target protein when administered to the human or animal circulation in the complex of the invention preferably is increased compared to its half-life when administered in its free form (e.g. only bound to small ions but not to the conjugate of the invention) by a factor of at least 1.1, more preferably a factor of at least 1.2, at least 1.3, at least 1.5, at least 1.8, at least 2, at least 3, at least 5, and most preferably a factor of at least 10.

The complex of the invention may be used in medicine. Preferably, the target protein of the complex has a pharmaceutical activity useful in the treatment or prevention of certain diseases. More preferable, the target protein exerts its pharmaceutical activity in the circulation of the human or animal patient. Preferably, the disease to be treated or prevented is caused by defects in or decreased amounts or absence of a natural protein in the human or animal body. In particular, the disease may be caused by an inherited or acquired defect in the gene of a particular protein. Particular examples of such diseases are hemophilia A and hemophilia B. For example, the target protein may be factor VIII and the complex may be used in the treatment of hemophilia A, or the target protein may be factor 1× and the complex may be used in the treatment of hemophilia B.

Thus, in another aspect, the invention relates to a pharmaceutical composition comprising the complex of the invention. The pharmaceutical composition may further comprise any pharmaceutically acceptable carriers, diluents and/or excipients suitable for administering the complex to a human or animal patient. They are preferably administered parenterally, particularly by injection, for example intravenal or intraarterial injection.

Preferably the pharmaceutical composition comprises the complex of the invention in a concentration of from 1 pM to 1 mM or in an amount of from 1 pg to 500 mg, preferably 100 ng to 500 mg per single dose.

The complex of the invention may be prepared by contacting the target protein with the conjugate. The target protein may be contacted with the conjugate in a suitable solvent, preferably an aqueous solvent, for example water with a physiologic salt concentration and a physiologic pH in the range of from about 6.0 to about 8.5. However, the target protein may also be contacted with the conjugate in other solvents and under other conditions as long as the solvent and the conditions do not inhibit the formation of the complex.

In a further aspect, the invention is directed to the conjugate used for forming the complex. Special embodiments of the conjugate are described above in the sections dealing with the complex of the invention and it is referred to the above disclosure. Furthermore, the invention pertains to the use of a respective conjugate for preparing the complex according to the present invention. Preferably, said complex is prepared ex vivo.

Furthermore, the invention is directed to a method for increasing the half-life of a target protein in the circulation of a human or animal, comprising the step of contacting the target protein with the conjugate of the invention comprising a polymeric moiety and a binding moiety having a binding capacity to the target protein. After contacting the target protein with the conjugate, they form a complex. The complex may be formed directly upon contact or may be formed only upon transferring the target protein and the conjugate into an appropriate environment such as, for example, the circulation of a human or animal. Preferably, the target protein is contacted with the conjugate prior to administration to the human or animal body. In certain embodiments, the method is performed outside of the human or animal body.

The target protein may be contacted with the conjugate in a molar ratio of from 10:1 to 1:10000, preferably 1.2:1 to 1:100, and more preferably 1:1 to 1:10.

In the following, a preferred embodiment wherein the target protein comprises a heparin binding site is described in more detail.

A number of proteins involved in hemostasis bind to heparin or heparinoids. Amongst others, antithrombin, thrombin, von Willebrand factor, tissue plasminogen activator, factor VIII and factor IX bind heparin or heparin-like, acidic polysaccharides. The binding of heparinoids is mediated by so-called heparin binding domains expressed on the protein surface. The relatively high affinity of the heparinoid-protein interaction allows non-covalent modification of the heparin-binding proteins by heparinoids or chemically modified derivatives thereof.

As can be seen in the crystallographic structure of blood clotting factor VIII (see FIG. 1), heparan sulfate proteoglycan at position S558-Q565 is free for coupling to hydroxyalkyl starch.

Additionally, the low-density lipoprotein-receptor-related protein (LRP) bound to FVIII at position R484-F509 and/or E 1811-K1818 could be targeted for coupling (see FIG. 1).

In general, a modification of the biological mechanism is intended such as the catabolism and/or the activity, resulting in an increase or inhibition of specific molecule-molecule interactions. As a resulting consequence the half-life of the target protein is increased.

Binding ligands could be acidic, negatively charged polysaccharides such as, for example, heparin or other binding substances such as mimetic peptides, nucleic acid and/or organic molecules.

As binding molecules, these highly affine ligands could be used as such or with special modifications. In order to form the conjugate, they have to be coupled to polymers such as hydroxyalkyl starch (HAS/HES), polyethylene glycol (PEG), oligosaccharides, and the like.

Acidic polysaccharides can be heparin, heparan sulfate, proteoglycans, glycosaminoglycans and other acidic, preferably anionic carbohydrate polymers, like for example fucoidan, sulphated fucans or galactans.

Heparin-like acidic oligosaccharides, called ‘heparinoids’, are highly acidic, e.g. highly sulfated polysaccharides. Heparinoids are ubiquitously found in most species, at least heparan sulfate is found in virtually all animals.

Heparin typically consists of repeating disaccharides built of D-glucosamine and either D-glucuronic acid or L-iduronic acid. D-gulcosamine can exist as N-acetylated or N-sulfated aminohexose and can be further sulfated at C6 or C3. The polysaccharide-chain can be of different lengths with molecular weights typically ranging between 3 kDa and 40 kDa, with a reducing and a non-reducing end. The reducing end can be used for a number of chemical reactions, i.e. for coupling to proteins, carbohydrates and other reaction partners. The structure of heparin, heparin sulfate is thoroughly described in ‘Heparin-Binding Proteins’ by H. Edward Conrad, Academic Press, 1998.

Heparin is used commercially for anticoagulation in-vivo and for surface coating of medical devices.

An outstanding property of heparin and heparinoids is their strong interaction with a number of so-called heparin-binding proteins. More than 100 proteins have been found to bind to heparinoids. Amongst them, not only coagulation factor VIII or IX, but a number of other proteins not only related to coagulation and fibrinolysis, specifically and strongly bind to heparinoids. The most prominent and typical example of a heparin-binding proteins is antithrombin.

Other heparin binding proteins are for example: antithrombin, thrombin, von Willebrand factor, tissue plasminogen activator, factor VIII, factor IX, vitronectin, protein C inhibitor, tissue factor pathway inhibitor, platelet factor 4, histidine-rich glycoprotein, thrombospondin, urokinase, fibronectin, fibroblast growth factors, hepatocyte growth factor, lipases, apolipoprotein B and apolipoprotein E.

Amongst them, a number of proteins related to coagulation and fibrinolysis specifically and strongly bind to heparonoids. The most prominent and typical example of a heparin-binding proteins is antithrombin.

Heparin-binding proteins exhibit at least one so-called heparin-binding domain. The nature and common structural features of heparin-binding domains has been described previously e.g. by H. E. Conrad, ‘Heparin-Binding Proteins’, Academic Press, 1998.

A striking property of the heparin-protein binding is its relatively high affinity. The binding strength of the heparinoid-protein binding usually is expressed by its dissociation constant Kd, as described above.

The synthesis of artificial acidic polysaccharides for non-covalent binding to the heparin binding site of proteins may result in even higher binding affinities between the ligands, i.e. the artificial acidic polysaccharide and the binding protein.

Thus, acidic polysaccharides like heparinoids and specifically synthesized, artificial acidic polysaccharides are ideal ligands for high-affinity, non-covalent binding to heparin-binding proteins e.g. heparin-binding proteins involved in hemostasis like human or recombinant coagulation factor VIII, human or recombinant coagulation factor IX, human or recombinant von Willebrand factor and other binding proteins.

Heparin and heparan sulphate are abundant molecules, which interact with a large variety of different binding partners and therefore display some kind of promiscuous binding behaviour.

Replacement of heparin by other heparinoids could trigger and optimize the binding affinity and specificity for the target protein. Furthermore, the risk of containing infectious agents such as viruses or prions could be reduced by switching for example to an anionic polysaccharide artificially synthesized or of plant origin, or using heparin mimetic peptides.

One possible alternative for heparin are fucoidans or sulphated fucans.

Fucoidans are sulfated plant polysaccharides (Mw average 20.000 Da) abundant in coastal waters all over the world. They are mainly found in brown algae (Phyaeophyceae) and marine invertebrates. Fucoidans from brown algae comprise complex, highly branched and sulphated polysaccharide structures, whereas sulphated fucans from marine invertebrates are linear homopolymers of fucose, comprising only a single linkage type and showing a regular, repeating oligosaccharide structure, but a varying sulphation pattern (Pomin & Mourao, 2008; reviewed in Berteau & Mulloy, Glycobiology 13, 2003). Fucoidan and sulfated fucans are suggested as heparin alternatives, because they share many biological activities (Berteau & Mulloy, 2003), due to their ability to imitate patterns of sulphate substitution on glycosaminoglycans and other sulfated glucans. Like heparin, they can act as modulators of coagulation (Chargaff et al., 1936) and can affect many biological activities, such as inflammation, cell proliferation and adhesion, viral infection and fertilization (Boisson-Vidal et al., 1995). Due to their vegetable origin, fucoidans are less likely to contain infectious agents such as viruses or prions. This makes them a desirable alternative for heparin as i.e. fucoidan-polymer conjugates, for non-covalent binding to heparin-binding proteins for a half-life prolongation purpose.

Independent of heparin and heparinoids there are a lot of possible species as highly specific and affine non-covalent binding partners for the said target proteins. These binding species include: specifically binding peptides (sequences derived for example from antibody recognition domains) or mimetic peptides, non-neutralizing antibodies, small molecules or DNA/RNA fragments (e.g. aptamers).

Aptamers are nucleic acid species that have been engineered through repeated rounds of in vitro selection or equivalently, SELEX (systematic evolution of ligands by exponential enrichment) (Tuerk. & Gold (1990) Science) to bind to various molecular targets such as small molecules, proteins, nucleic acids, and even cells, tissues and organisms. (Ellington & Szostak (1990) Nature). Aptamers are useful in therapeutic applications as they offer molecular recognition properties that rival that of the commonly used biomolecule, antibodies, namely high specificity and affinities in the low nanomolar to the picomolar range. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

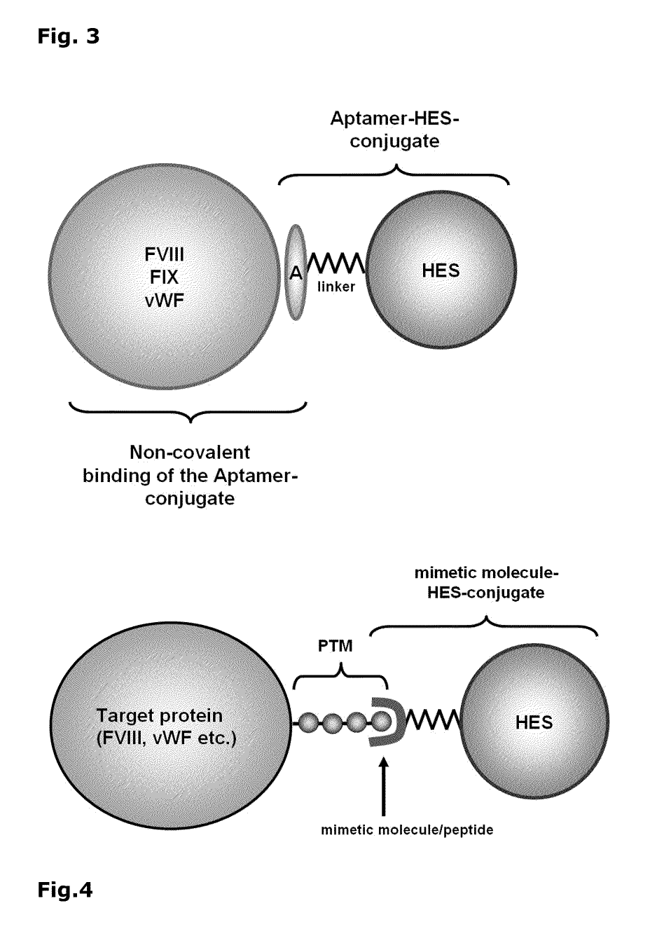

Aptamers are here suggested as non-covalent binding partners to a target protein within an aptamer-polymer-conjugate (see FIG. 3) because they exhibit a very specific and highly affine binding to it. Due to their in vitro origin, aptamers are completely free of infectious agents such as viruses or prions, making them a desirable component for the half-life prolongation approach.

A slightly different approach could focus on posttranslational modifications (PTMs, such as glycosylations) as possible targets for specific interactions with non-covalent binding partners for half-life prolongation.

As an example, the glycosylated FVIII/vWF complex is given here. The desialylation of FVIII/vWF for example is responsible for fast plasma clearance by asialoglycoprotein recpetor (ASGP-R; Sodetz et al., JBC 1977 & 1978). Based on the knowledge of these well characterized ASGP-R interactions (Meier 2005) a ASGP-R mimetic peptide could be generated and synthesized and attached via a short linker to a polymer, e.g. a HES/HAS-moiety (see FIG. 4). This conjugate could then be used to block the ASGP-R binding site and inhibit receptor-mediated clearance by shielding effects or steric hinderince, thus prolonging plasma half-life.

Another example for using a mimetic peptide based on a specific non-covalent interaction between a target protein and an enzyme, is the interaction between Elastase and G-CSF:

Elastase, also known as neutrophil elastase (NE) or human neutrophil elastase (HNE) specifically binds to G-CSF (Hunter et al., 2003). Based on the amino acid sequence of the elastase binding region a mimetic peptide could be generated, synthesized and attached to a polymer, e.g. via a short linker to a HES/HAS-moiety to prolong the half-life of G-CSF.

Based on the existing X-ray structure of G-CSF (Hill et al., PNAS (90) 1993) and several known non-neutralizing anti-G-CSF antibodies (Layton et al., JBC (266) 1991), mimetic peptides could be generated mimicking the target event in order to perform non-covalent coupling of a polymer to the target protein, G-CSF in this example in order to prolong half-life of G-CSF pharmaceutical drug.

In specific embodiments, the invention is directed to a complex comprising at least one protein with a heparin binding site and at least one heparin or a heparin-like molecule, wherein the heparin or heparin-like molecule is covalently bound to hydroxyalkyl starch.

The protein may be selected from factor VIII, antithrombin, thrombin, von Willebrand factor, tissue plasminogen activator, and factor IX.

The hydroxyalkyl starch may be hydroxyethyl starch. The molecular weight of the hydroxyalkyl starch is for example in the range of 100 to 300 kD. Besides coupling at least one heparin or heparin-like molecule, also at least two heparin molecules or heparin-like molecules may be covalently bound to one hydroxyalkyl starch molecule.

Furthermore, the invention provides a method for preparing a complex as described above, comprising the steps of:
coupling at least one heparin molecule or heparin-like molecule to at least one hydroxyalkyl starch to get a heparin-hydroxyalkyl starch conjugate
incubating the conjugate with a protein having a heparin binding site.

In the method, the hydroxyalkyl starch may be hydroxyethyl starch. The at least one heparin molecule or heparin-like molecule may be bound to the at least one HAS molecule through a bifunctional linker, which for example can be homobifunctional or heterobifunctional.

At least one of the heparin or heparin-like molecule and hydroxyalkyl starch may be modified by the following steps:
oxidizing to introduce aldehyde groups
reductive amination.

In one embodiment, at least two heparin molecules or heparin-like molecule are bound to one hydroxyalkyl starch molecule.

The invention is further directed to a heparin-hydroxyalkyl starch or a heparin-like molecule-hydroxyalkyl starch conjugate; a pharmaceutical composition comprising the complex described above, wherein the protein may be factor VIII; and the use of the complex described above for the preparation of a medicament for treating bleeding disorders.

In one embodiment the complex does not comprise at least one protein with a heparin binding site and at least one heparin or a heparin-like molecule, wherein the heparin or heparin-like molecule is covalently bound to hydroxyalkyl starch.

In one embodiment a method for preparing the complex does not comprise the steps of:
coupling at least one heparin molecule or heparin-like molecule to at least one hydroxyalkyl starch to get a heparin-hydroxyalkyl starch conjugate and
incubating the conjugate with a protein having a heparin binding site.

In one embodiment the conjugate is not a heparin-hydroxyalkyl starch conjugate or a heparin-like molecule-hydroxyalkyl starch conjugate.

### EXAMPLES

### Example 1

### Synthesis Chemistry: HES-Heparinoid-Conjugates

One approach for the in vivo half-life prolongation of the recombinant factor VIII is based on hydroxyethyl starch as macromolecular carrier which is attached non-covalently via a selective binding site on factor VIII. For this non-covalent attachment heparin, its derivatives or its mimetics can be used as factor VIII binding molecules. HES is bound to these heparinoids to form a large complex which covers and protects part of the protein surface.

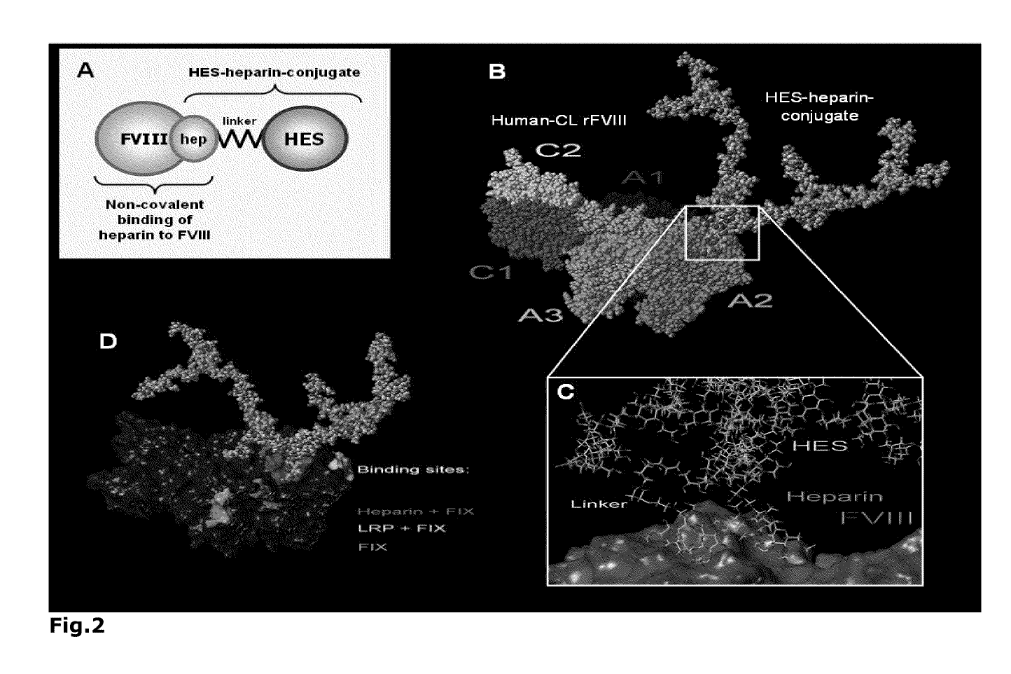

The proof of this principle was evaluated by factor VIII binding studies by surface plasmon resonance followed by an extensive in vivo study using a covalent 1:1 conjugate between the heparinoid and hydroxyethyl starch. FIG. 2 shows the three-dimensional structure of factor VIII in complex with a 1:1 heparinoid-HES conjugate.

To obtain this 1:1 conjugate, both heparin and HES were chemically modified only at one position with complementary functional groups, which allowed a selective conjugation. Both molecules have exactly one unique functional group, the aldehyde/semiacetal group at the reducing end of the polysaccharide chain, which is chemically different to all other functionalities in these molecules. Thus, the developed synthesis strategy was based on derivatizations of the reducing ends.

Hydroxyethyl starch may be separated into two different size fractions and as factor VIII-binding molecule a low molecular weight heparin (LMWH), e.g. Enoxaparin, can be used. The 1:1 conjugation of C1-activated hydroxyethyl starches with C1-modified LMWH is based on a Michael-addition of a free thiol with a maleimide functionality.

### Hydrolysis and Fractionation of HES450/0.7

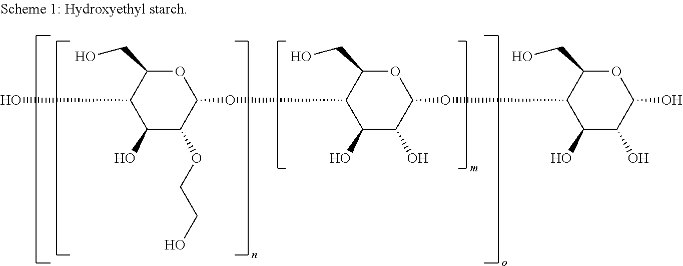

30 g HES450/0.7 (hydroxyethyl starch having a weight average molecular weight of 450 kDa and a molar degree of hydroxyethyl substitution per glucose unit of 0.7) were dissolved in 300 ml water and 1 M hydrochloric acid was added slowly until the pH reached 2.0. The solution was then heated up to 80° C. and stirred vigorously for 16 h. Afterwards the solution was cooled down below 50° C. and brought to pH 5-6 by slowly adding 1 M sodium hydroxide solution. The partially hydrolyzed HES450/0.7 was fractionated by sequential tangential-flow ultrafiltration steps using peristaltic pumps and PES ultrafiltration membranes with different molecular weight cut-offs (e.g. MWCO=100, 30, 10, 5 kDa).

Used HES fraction: HES25/0.7 (Mw=25 kDa) and HES 54/0.7 (54 kDa)

### Synthesis of C1AminoHES

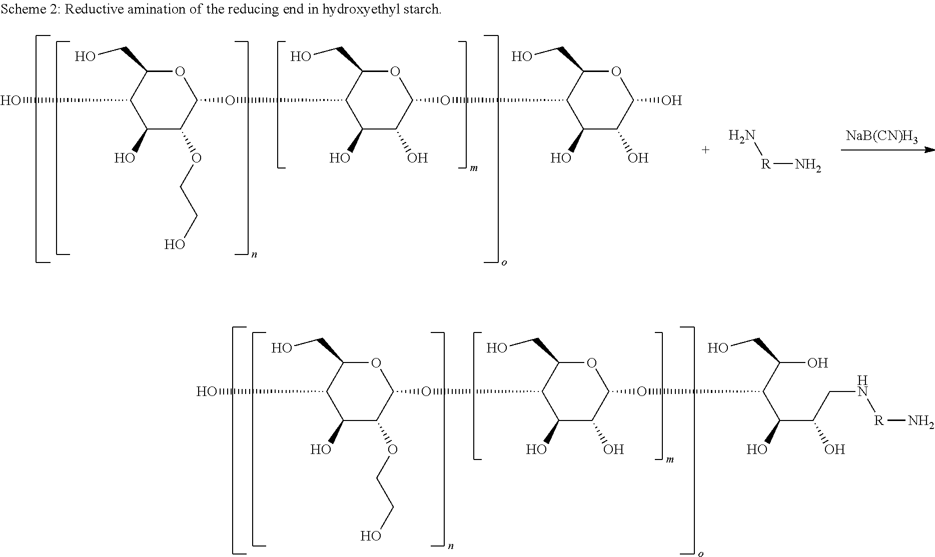

200 mg HES25/0.7 were dissolved in 12 ml 50 mM borate buffer pH 8.2 and 0.168 ml 1,3-diaminopropane and stirred for 1 h at 50° C. Then 0.126 g sodium cyanoborohydride were added and stirred at 50° C. for 3 days. The product was purified by ultrafiltration via a 1 kDa ultrafiltration membrane and afterwards the product was lyophilized. The obtained white solid is stored at room temperature.

### Synthesis of C1AminoHES Via OxHES

OxHES was obtained by an oxidation with iodine in sodium hydroxide (based on PhD thesis Michele Orlando, Giessen, Germany 2003).

1 g HES25/0.7 was dissolved in water and 9.0 ml of a 0.1 N iodine solution followed by 1.4 ml of a 1 N NaOH solution were added. The solution was stirred overnight. The reaction product was purified by ultrafiltrated via a 1 kDa PES membrane. The final solution was passed through a cation ion exchange resin (Amberlite IR-120 H+) and lyophilized. The obtained product was dried to remove most residual water.

0.1 g of the obtained OxHES were dissolved in 1.5 ml DMSO. 115 mg HOBt and 36.7 mg DMAP were added. Then 190 mg HATU and afterwards the 1,3-diaminopropane were added and the solution was stirred for 1 h at 37° C. Another 190 mg HATU were added and the solution was stirred overnight at 37° C.

The product was precipitated by adding 25 ml acetone/ethanol 1:1 and the precipitant was washed with acetone/ethanol 1:1. The pellet was dissolved again in 2 ml DMSO and the product was again precipitated by adding 25 ml acetone/ethanol 1:1, followed by another washing step.

The remaining product was dissolved in 100 ml water and further purified by an ultrafiltration via a 1 kDa membrane. Afterwards the product was lyophilized.

### Synthesis of C1MalemideHES

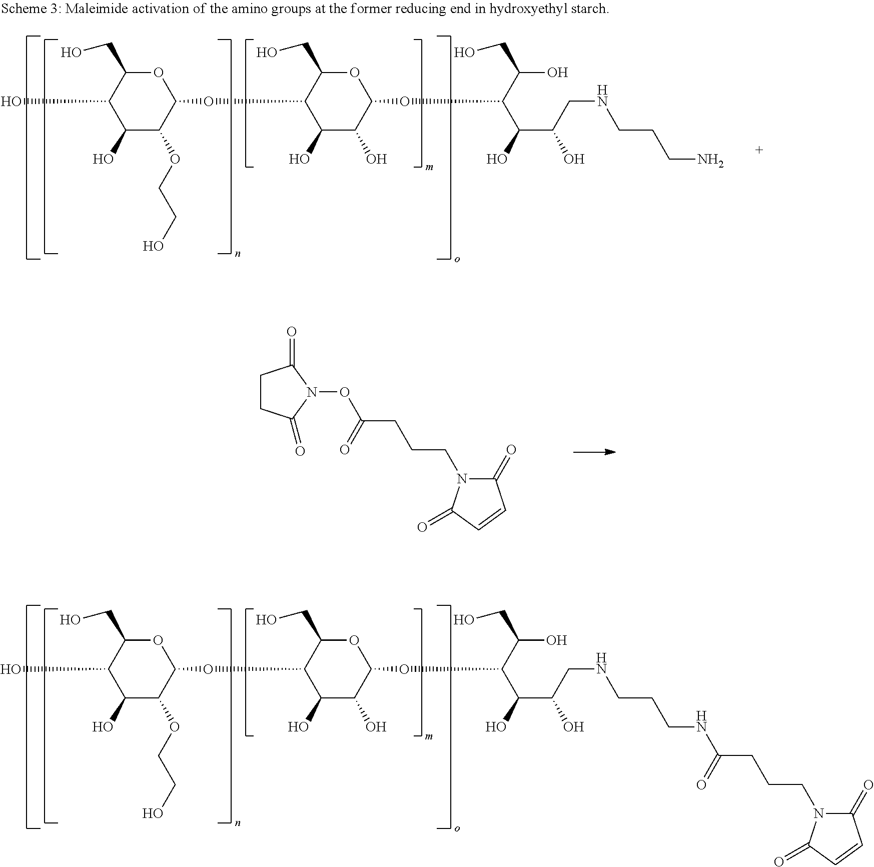

500 mg C1AminoHES was dissolved in 15 ml 1-methyl-2-pyrrolidinone (NMP). A solution of 140 mg N-[g-maleimidobutyryloxy] succinimide ester in 10 ml NMP and 85.6 μl N,N-diisopropylethylamine were added. The solution was stirred or shaken for 90 min. The product was precipitated by pouring the solution in 100 ml acetone/ethanol 1:1 and cooling down to −20° C. The precipitant was centrifuged down, the supernatant decanted and the pellet was washed two more times with acetone/ethanol 1:1 and centrifuged down. The product was purified by ultrafiltration via a 1 kDa ultrafiltration membrane and lyophilized. The obtained solid product was stored at −20° C.

### Chemical Modification of a Low-Molecular Weight Heparin (LMWH)

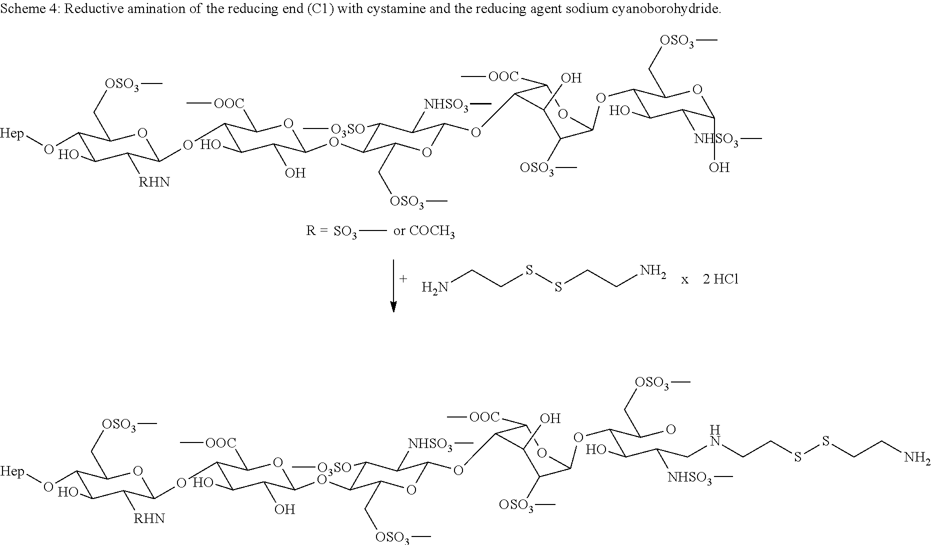

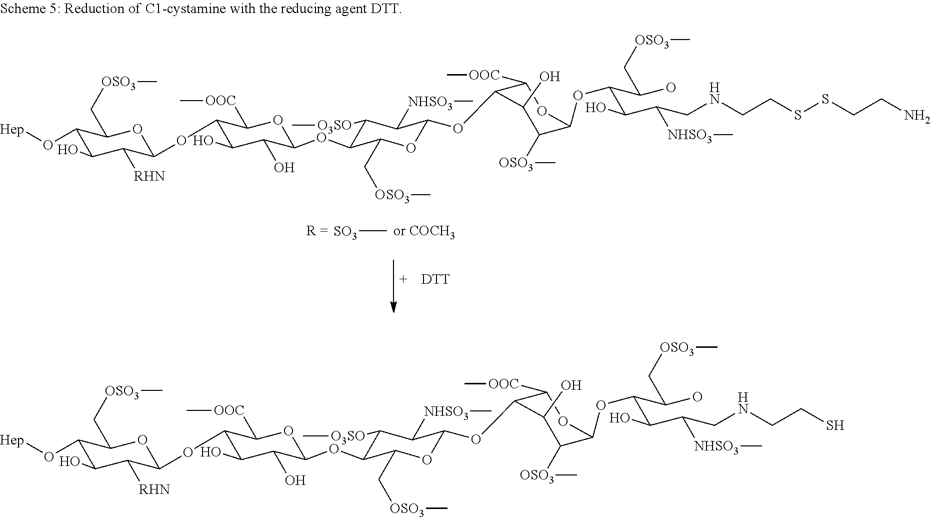

250 mg lyophilized LMWH were dissolved in 12 ml of a 0.3 M phosphate buffer pH 5.0 containing 1 M sodium chloride. 1.34 g cystamine dihydrochloride were added and the solution was stirred or shaken for 1 h at 40° C. Then 0.374 g sodium cyanoborohydride were added and the mixture was stirred or shaken for 3 days at 40° C.

The product was purified by a size exclusion chromatography using a 50 mM phosphate buffer pH 6.5 with 100 mM NaCl as mobile phase on a HiLoad 26/60 Superdex 30 prep grade column and a flow of 4.4 ml/min. The unified product fractions were directly employed in the next step.

To the obtained fractions 91.8 mg DL-1,4-dithiothreitol (DTT, 0.6 mmol, 10 eq) were added and the solution was stirred overnight at room temperature. The product was purified by a size exclusion chromatography using water as mobile phase on a HiLoad 26/60 Superdex 30 prep grade column and a flow of 4.4 ml/min. The product fractions were unified and lyophilized. The obtained white solid was stored at −20° C.

Conjugation of C1 Maleimide HES and thioLMWH

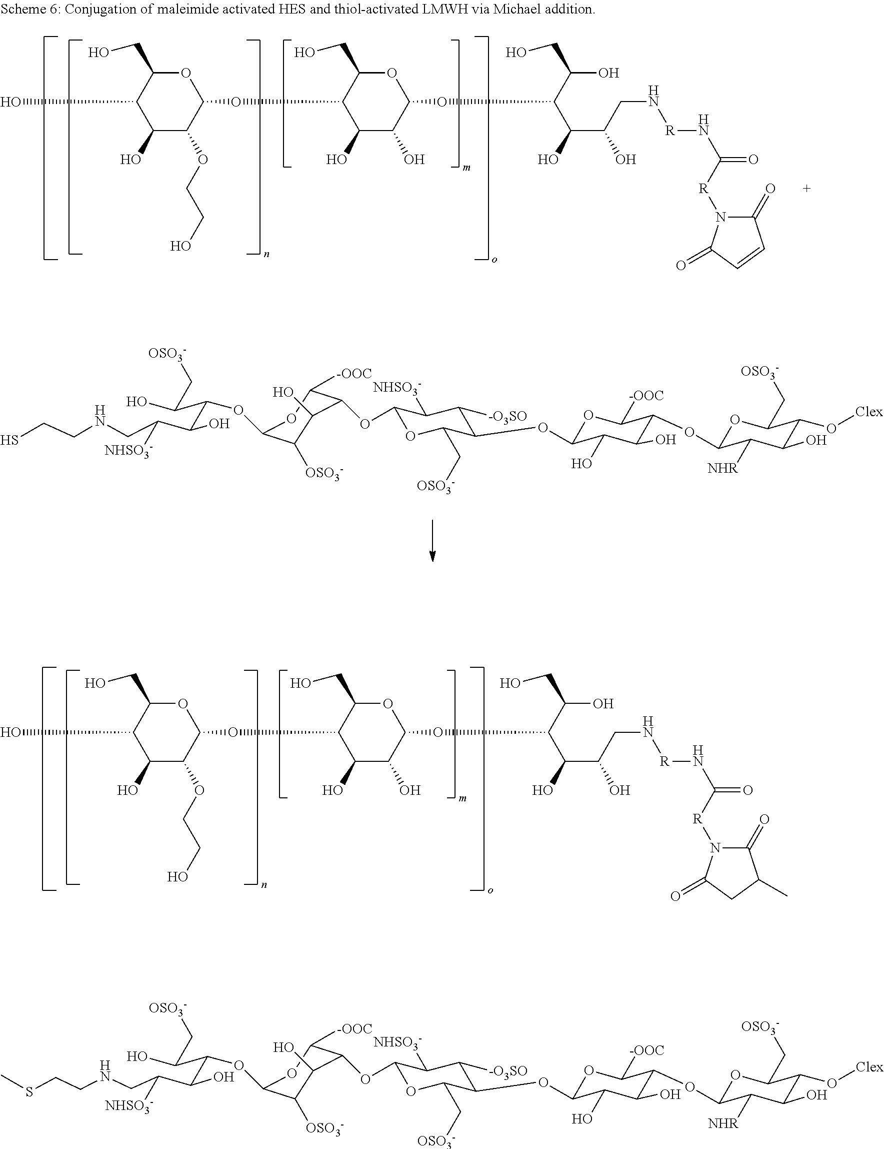

32.2 mg of C1LMWH-cysteamine and 420 mg of C1MaleimideHES were dissolved in total 14.5 ml 100 mM phosphate buffer pH 7.5, mixed and shaken overnight at 37° C.

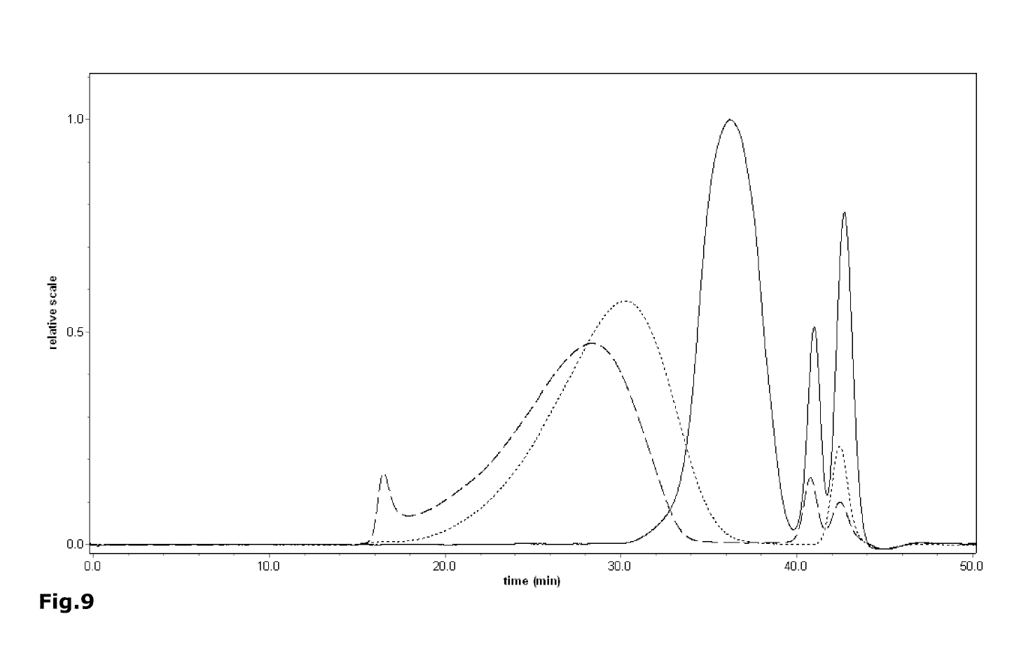

The LMWH-HES conjugate was purified on a HiPrep 16/10 Q FF column (GE Healthcare) with the 50 mM TrisHCl-buffer pH 7.5 for binding and 50 mM TrisHCl-buffer pH 7.5 with 1 M NaCl for elution using a linear gradient with a flow of 5 ml/min. The fraction eluting at conductivities greater than 30 mS/cm have been further purified on a HiLoad 26/60 Superdex200 prep grade column (GE Healthcare) using a 50 mM phosphate buffer pH 6.5 with 0.1 M NaCl and a flow of 4 ml/min. The product was desalted on a HiLoad 26/60 Superdex200 prep grade column (GE Healthcare) with water as mobile phase and a flow of 4 ml/min. The chromatograms are shown in FIG. 9. The obtained product was further lyophilized and stored at −20° C.

### Heparin-HES

Examples 1 and 2 are composed of non-covalent complexes of coagulation factor VIII and HES-heparin-conjugates (see FIG. 2). FVIII possesses a defined heparin-binding site at the A2-domain and is able of binding heparin and heparinoids (such as low molecular weight heparins (e.g. Clexane) or fucoidans) with high affinity. The heparin or heparinoid is covalently attached via a short linker region to a HES-moiety. Non-covalent binding of this HES-heparin-conjugate to FVIII mediates the half-life prolongation by shielding effects.

### Surface Plasmon Resonance Binding Studies

Surface Plasmon Resonance (SPR) binding studies were performed using a Biacore T100 system (GE Healthcare Europe GmbH, Freiburg, Germany). The detection system is based on surface plasmon resonance to monitor molecular interactions in real time (Malmquist and Karlsson, 1997; Rich and Myszka, 2003; Piliarek 2009).

Binding reactions cause a change in the surface plasmon resonance, which are detected optically and measured in resonance units. One thousand RU correspond to a 0.1° shift in the surface plasmon resonance angle and a surface concentration change of about 1 ng/mm² for the average protein (Johnsson et al., 1991).

Prior to analysis, human recombinant blood clotting factor VIII, human-CL rFVIII, and human recombinant blood clotting factor IX, rFIX, were loaded onto a NAP5 column for buffer exchange with 10 mM NaAc pH 5/pH 5.5 and were then immobilized on the dextran matrix of a CM5 biosensor chip using the amine coupling kit as prescribed by the supplier. A reference channel was activated and blocked in the absence of protein.

Association of the analytes HES-heparin conjugates and Fucoidan were assessed in 10 mM Hepes, 150 mM NaCl and 0.005% (v/v) Tween 20 (pH 7.4) at 25° C. with a flow rate of 10 μl/min for 3 min. Dissociation was allowed for 15 min. in the same buffer flow.

After dissociation the biosensor chip was regenerated by injection of 800 mM NaCl for 1 min. Binding of HES-heparin conjugates and Fucoidan to the human-CL rFVIII and human rFIX-coated channels were corrected for non-specific binding to the control channel.

For determination of binding constants, increasing concentrations of the analyte were applied to the immobilized human recombinant blood clotting factor VIII. Binding profiles were evaluated using the Biaevaluation software to fit kinetic titration data. The kinetic data (association rate constant [kₐ], dissociation rate constant [k_{d}]) as well as affinity data (dissociation equilibrium constant K_{d}) were calculated by computer-based evaluation of the binding data.

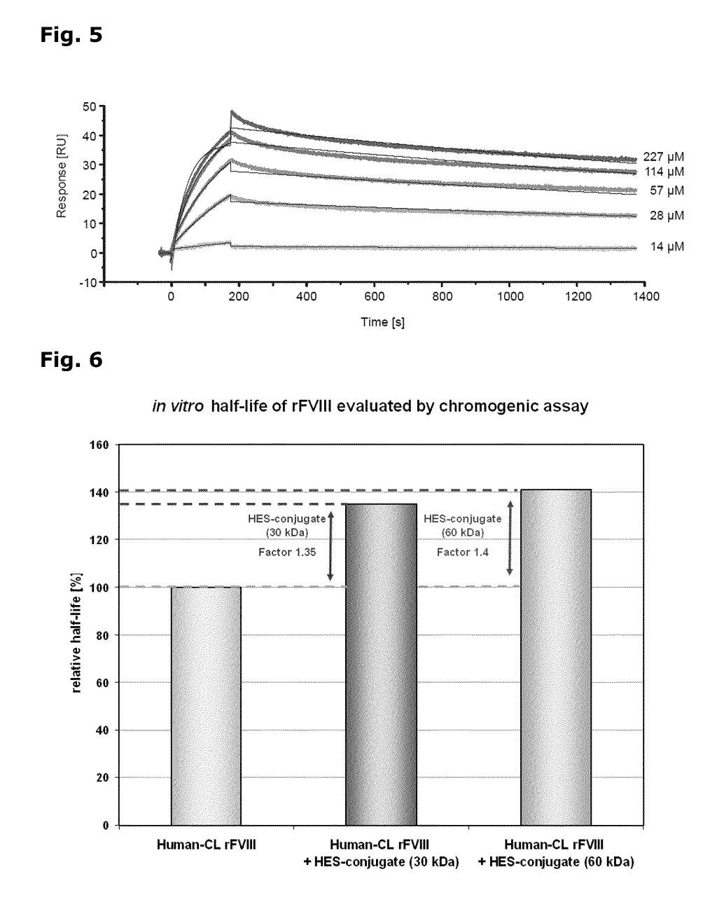

Binding studies of HES-heparin-conjugates to rFVIII were performed using the SPR method. Human-CL rFVIII was immobilized to the sensor chip and the association and dissociation of the 30 kDa HES-heparin-conjugate was monitored via SPR at increasing conjugate concentrations (14-227 μM). FIG. 5 shows a typical set of binding curves, demonstrating binding of the HES-heparin-conjugate to human-CL rFVIII for all concentrations. Software-based analysis of the binding curves yielded a dissociation constant K_{d} of 6 (±0.6) 10⁻⁷ M, clearly demonstrating a strong binding of the HES-heparin conjugate to human-CL rFVIII.

### In Vitro Stability Study

An in vitro stability study was performed to simulate the pharmacokinetics study in an artificial system and to determine the effect of binding of the HES-heparin-conjugates on FVIII stability. Therefore, 4 IU of human-CL rFVIII were mixed in a 1:2 nominal molar ratio with the two different HES-conjugates (HES(30 kDa)- and HES(60 kDa)-conjugate, respectively), solved in 40 μL buffer and added to 1.7 mL of FVIII deficient plasma (Coachrom Cat. no. FDP08-10, Lot no. D8-22), which mimics the whole blood volume of a haemophilic mouse comprising a body weight of 20 g. Three different approaches (human-CL rFVIII as reference, human-CL rFVIII complexed with HES-conjugate (30 kDa), human-CL rFVIII complexed with HES-conjugate (60 kDa)) were incubated at 37° C. and samples were taken and directly frozen at −80° C. each after defined time interval.

After thawing, the samples were analysed for FVIII activity by a chromogenic assay (Coachrom Diagnostica GmbH, order #221402; lot #72502-PK:6). This chromogenic assay determines the FVIII:C activity in two consecutive steps: addition of activated FIX (FIXa), phospholipids and calcium to the FVIII samples activates FX to FXa and the latter subsequently cleaves an added FX-substrate yielding a chromophore which can be quantified spectrophotometrically. Under appropriate assay conditions the relationship between FXa formation (and thus chromophor generation) and FVIII concentration is linear.

The FVIII-deficient plasma and incubation at 37° C. mimicked the haemophilic mouse model as described above. The plasma samples were subsequently analysed using a chromogenic assay testing for FVIII:C activity. The resulting data were converted into half-life using the sigma plot computation software. A direct comparison of the relative half-life (in %) of human-CL rFVIII without HES-conjugates with human-CL rFVIII complexed with HES-conjugate (60 kDa) showed a 1.4 times prolonged in vitro plasma half-life. Complexation with HES-conjugate (30 kDa) revealed a half-life prolongation of factor 1.35, clearly indicating a half-life prolonging effect of both HES-conjugates non-covalently bound to FVIII in vitro (see FIG. 6).

### Pharmacokinetics Study in Hemophilic Mice

Pharmacokinetics studies in haemophilic mice were performed as follows:

12 weeks old C57 BL/6 FVIII knockout mice of mixed sex and a body weight of 20-25 g were used for this study.

The following FVIII variants were to be tested and prepared as described below:

Human-CL rFVIII as reference, human-CL rFVIII complexed with a 60 kDa or a 30 kDa HES-heparin-conjugate and buffer as negative control. Human-CL rFVIII was complexed with the HES-heparin-conjugates in a 1:2 nominal molar ratio and sterile filtrated, as well as the reference human-CL rFVIII without HES-heparin-conjugates and the buffer negative control.

40 μL comprising 4 IU of FVIII (consistent with around 200 IU/kg) were injected as a single administration through the tail vein into the haemophilic mice. Samples were taken pre- and post-injection and at defined time intervals after administration (with groups of 5 mice per sampling time and substance). FVIII:C activity was determined using a chromogenic assay commercially available from Coachrom Diagnostica GmbH, Vienna, Austria and the pharmacokinetics of FVIII calculated from these results and statistically analysed (including the t-test) using the sigma plot computation software (Systat Software GmbH, Germany) to obtain the half-life times (T₁₁₂).

The following FVIII variants were tested in comparison for their plasma half-life in haemophilic mice:

Human CL-rFVIII as reference (group 1), human-CL rFVIII complexed with a 60 kDa HES-conjugate (group 2), human-CL rFVIII complexed with a 30 kDa HES-conjugate (group 3) and buffer as negative control (group 4). In general, 25 haemophilic mice per substance were injected with 4 IU of rFVIII in a total injection volume of 40 μL per mouse. Samples were taken pre- and post-injection and at defined time intervals after administration, with 5 mice per sampling time and substance (exception: group 3, where only 19 mice in total were used due to material limitations).

The samples were tested for FVIII:C activity and the data analysed regarding the pharmacokinetic parameters as described above, the results are summarized in the following table:

TABLE 1

| | | | | Factor for | |
|---|---|---|---|---|---|
| | | | | half-life | |
| | | | | prolongation | P-value |
| | | | Relative | compared to | of |
| Group | Tested | No. of | half-life | human-CL r | statistical |
| # | substance | mice | [%] | FVIII | analysis |
| 1 | Human-CL | 25 | 100 | 1 | 0.017 |
| | rFVIII | | | | |
| 2 | Human-CL | 25 | 170 | 1.7 | 0.006 |
| | rFVIII-HES- | | | | |
| | conjugate | | | | |
| | (60 kDa) | | | | |
| 3 | Human-CL | 19 | 155 | 1.6 | 0.016 |
| | rFVIII-HES- | | | | |
| | conjugate | | | | |
| | (30 kDa) | | | | |

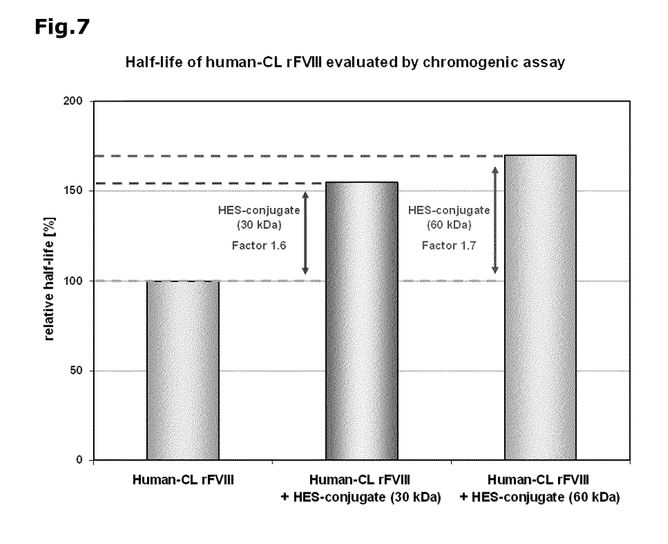

In direct comparison, human-CL rFVIII complexed with both the HES-conjugates shows a prolonged plasma half-life: for complexation with the smaller HES-conjugate (30 kDa) the plasma half-life is prolonged for the factor 1.6, complexation with the larger HES-conjugate (60 kDa) yields an 1.7 times increased plasma half-life (see FIG. 7).

These data clearly indicate a protective, half-life prolonging effect of the non-covalently bound HES-heparin-conjugates on FVIII in the hemophiliac mouse model, this effect being slightly more pronounced for the larger HES-heparin conjugate (60 kDa) than for shorter HES-heparin conjugates (30 kDa).

As a negative control, haemophilic mice were injected with buffer and, as expected, no FVIII activity was found by the chromogenic assay (data not shown).

### Fucoidan

In order to exemplify a possible alternative to heparin, fucoidan was tested for its binding properties to heparin-binding proteins, such as coagulation factor VIII and IX. SPR-based binding experiments demonstrate, that fucoidan could replace heparin within the half-life prolonging HES-heparin-conjugates for non-covalent binding to the target protein.

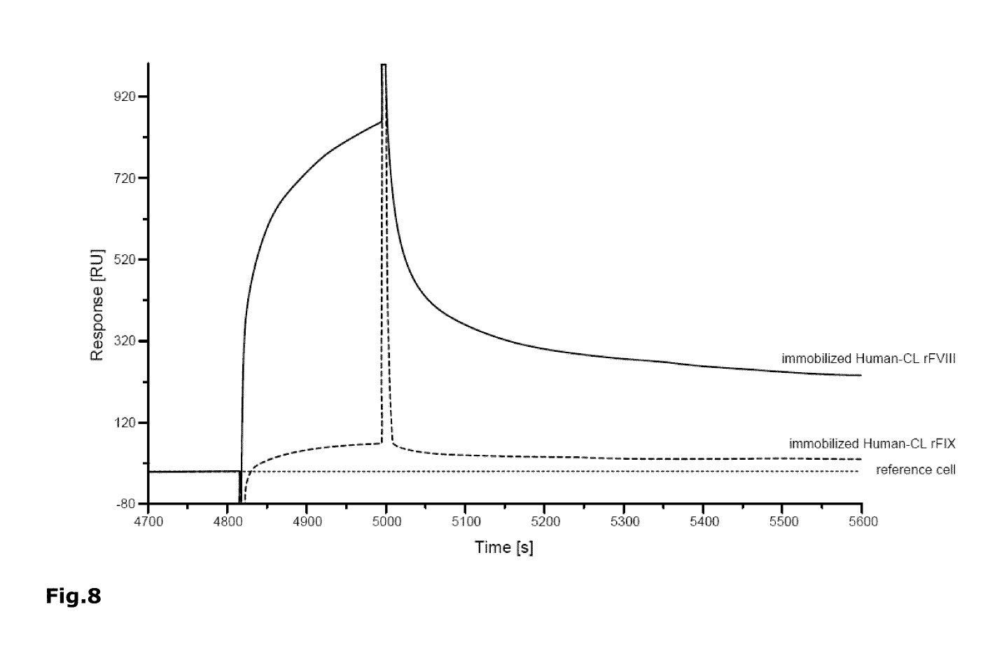

SPR-based binding studies evaluating fucoidan as a possible heparin surrogate were performed against human-CL rFVIII and rFIX as heparin-binding proteins. FIG. 8 shows the association and dissociation of fucoidan to rFVIII (straight line) and rFIX (dashed line) respectively, clearly demonstrating the binding of fucoidan to the probed proteins and consequently confirming its suitability as a possible heparin-surrogate.

1. A complex comprising at least one target protein and at least one binding molecule having a binding affinity for said target protein, wherein said molecule having a binding affinity is covalently or non-covalently bound to at least one water-soluble polymer.

2. The complex of claim 1, wherein the at least one binding molecule and the at least one soluble polymer form a conjugate, said conjugate being non-covalently coupled to the target protein.

3. The complex of claim 2, wherein the protein is selected from blood clotting proteins (plasma derived or recombinant) such as factor IX, factor VIII (wild-type and B-domain deleted), Factor VII/VIIa, thrombin, antithrombin, tissue plasminogen activator and von Willebrand factor (vWF), growth factors such as erythropoietin, colony-stimulating factors (CSFs) such as granulocyte stimulating factor (G-CSF), macrophage CSF (M-CSF) and granulocyte-macrophage CSF (GM-CSF), cytokines such as interleukins, protease inhibitors such as alpha-1-antitrypsin (A1AT), integrins, disintegrins, extracellular matrix proteins such as fibronectin and vitroncectin, metalloproteases such as matrix metalloproteases and ADAM/ADAMTS proteins, metalloproteases, apolipoproteins, transport proteins, hormones, inhibitory or regulatory acting proteins, and derivatives and mutants thereof.

4. The complex of claim 1, wherein the at least one binding molecule has a molecular weight below 50 kD, preferably below 10 kD.

5. The complex of claim 1, wherein the binding molecule is selected from the group consisting of peptide moieties such as RGD peptides and RGD binding domains, saccharide moieties such as heparins and heparan sulfates and heparin-like molecules, heparin-mimetic molecules, nucleic acid moieties, lipid moieties such as fatty acids, derivatives thereof and mimetics thereof.

6. The complex of claim 5, wherein the heparin-like molecule is a heparin-mimetic peptide.

7. The complex of claim 6, wherein the heparin-mimetic peptide comprises the amino acid motif X1-Y(SO3)X2-Y(SO3), wherein Y(SO3) is a sulphated tyrosine X1 is a negatively charged amino acids, serine, alanine or glycine and X2 is an aspartate, alanine or is absent.

8. The complex of claim 1, wherein the soluble polymer is selected from hydroxyalkyl starch (HAS), polyethylene glycol (PEG), polyvinylpyrrolidone (PVP), and dextran.

9. The complex of claim 1, wherein the molecular weight of the water-soluble polymer is in the range of 10 to 500 kD, 10 to 300 kD, 20 to 200 kD, 30 to 150 kD or 100 to 300 kD.

10. The complex of claim 2, the conjugate comprises
only one binding molecule and two or more polymers,
two or more binding molecules and only one polymer or
two or more binding molecules and two or more polymers.

11. A method for preparing a complex according to claim 1, comprising the steps of
coupling at least one binding molecule having a binding affinity for a target protein to at least one water-soluble polymer to form a conjugate,
incubating the conjugate with the target protein for which the molecule has a binding affinity.

12. The method of claim 11, wherein the at least one binding molecule is bound to the water-soluble polymer through a bifunctional linker.

13. The method of claim 11, wherein the complex is formed ex-vivo.

14. A conjugate of a molecule having a binding affinity for a protein and a water-soluble polymer.

15. A method for increasing the half-life of target proteins in the circulation of a human or animal, comprising the step of contacting the target protein with a conjugate according to claim 14.

16. A pharmaceutical composition comprising the complex of claim 1.

17. The use of the complex of claim 1 for the preparation of a medicament.

18. The complex of claim 1, wherein the at least one target protein is selected from factor IX and factor VIII (wild-type and B-domain deleted), the at least one binding molecule is a heparin-mimetic molecule and the at least one polymer is hydroxyethyl starch.
